(19) United States
(12) Patent Application Publication
(10) Pub. No.: US 2009/0318970 A1
(43) Pub. Date: Dec. 24, 2009

(54) Spinal Rod Connectors Configured to Retain Spinal Rods of Varying Diameters

(76) Inventors: Michael S. Butler, St. Charles, IL (US); Thomas J. Wegrzyn III, Chicago, IL (US)

Correspondence Address: BOWMAN & ASSOCIATES, 1016 3rd Avenue, SW, Suite #106, CARMEL, IN, 46032, US

(21) Appl. No.: 12/487,794

(22) Filed: Jun. 19, 2009

Related U.S. Application Data
(60) Provisional application No. 61/073,825, filed on Jun. 19, 2008.

(51) Int. Cl. A61B 17/70

(52) U.S. Cl. 606/264

(57) ABSTRACT

A spinal rod connector is configured to accommodate a range of spinal rod diameters. The present spinal rod connector may take the form of a spinal rod screw head, hook, spinal rod connector or a spinal rod holder. The spinal rod connector has spinal rod reception slots whose sides narrow towards a bottom of the slot. The narrowing of the sides provides a wedging effect such that a spinal rod is wedged into place during locking. The narrowing sides of the slots thus provide for the accommodation of varying rod diameters. The spinal connector also provides significant improvement in spinal rod torsional resistance compared to prior art radial shaped slots. In one form, the spinal rod reception slots are essentially V-shaped. However, other shapes following the principles of the present invention may be used and are contemplated. The width of the slot sides and the rate of curvature of the slot sides thus determine the range of spinal rod diameters that are accommodated by the spinal rod reception slots and therefore the spinal rod connector.

### RELATED APPLICATIONS

This patent application claims the benefit of and/or priority to U.S. Provisional Patent Application Ser. No. 61/073,825 filed Jun. 19, 2008, entitled “Spinal Rod Connectors Configured To Retain Spinal Rods of Varying Diameters” the entire contents of which is specifically incorporated herein by this reference.

### BACKGROUND OF THE INVENTION

1. Field of the Invention

The present invention relates to spine fixation components such as spinal screw assemblies, hooks and rod connectors for spinal rod applications and, more particularly, to spinal rod connectors for holding and retaining a spinal rod relative to a spinal screw.

2. Background Information

Spinal orthopedic assemblies and constructs such as spine plates, spinal bone screw assemblies, hooks and rod connectors for spinal rods and other devices (spinal components) have made a profound contribution to the correction of spinal deformities, accidents and other problems in the cervical as well as thoracic, lumbar and sacral spine. These and other spinal devices are typically fixed to vertebrae using vertebral bone screws. Vertebral bone screws are specially designed and manufactured bone screws that are placed into the bone of a vertebra. Vertebral bone screws placed in the vertebra offer superior strength and pull-out resistance as compared to other forms of fixation in spine surgery. The ability to achieve vertebral fixation has allowed surgeons to obtain more secure fixation of the spinal components involved, which permits more powerful correction of spine problems and reported better clinical outcomes.

In addition to other uses, bone screws provide a solid foundation for the attachment of spinal rods. Spinal rods are used for the fixation of a plurality of vertebrae for various situations. A spinal rod is held relative to the vertebrae by a spinal rod screw assembly. Various types of spinal rod screw assemblies are known such as those that allow for inter-operative adjustments in the coronal, transverse and sagittal planes. Certain spinal rod screw assemblies allow for various degrees of freedom of attachment of a spinal rod thereto from any direction, angle, and height. In all cases, however, the spinal rod screw assemblies hold a spinal rod via a spinal rod connector and are fixed to a vertebra via a spinal screw that is received by the spinal rod connector.

Spinal rods are made in various diameters for use in various situations. Because of this, spinal rod connectors are made in various dimensions in order to accommodate the diameter of the chosen spinal rod. Currently, spinal rod connectors are made to accept only one size (diameter) of spinal rod. Therefore, various sets of spinal rod connectors must be kept on hand for use with the various spinal rod diameters. This adds to the number of parts that must be kept in inventory.

In view of the above, there is a need for a single spinal rod connector or holder for a spinal rod screw assembly that can accommodate spinal rods of various diameters.

### SUMMARY OF THE INVENTION

The present invention is a spinal rod connector that is configured to accommodate spinal rods of varying diameters. More particularly, the present spinal rod connector is configured to accommodate a range of spinal rod diameters. The present spinal rod connector may take the form of a spinal rod screw head, hook, spinal rod connector or a spinal rod holder (collectively, “spinal rod connector”).

The present spinal rod connector has spinal rod reception slots whose sides narrow towards a bottom of the slot. The narrowing of the sides provides a wedging effect such that a spinal rod is wedged into place during locking. The narrowing sides of the slots thus provide for the accommodation of varying rod diameters. The present invention also provides significant improvement in spinal rod torsional resistance compared to prior art radial shaped slots.

In one form, the spinal rod reception slots are essentially V-shaped. However, other shapes following the principles of the present invention may be used and are contemplated. The width of the slot sides and the rate of curvature of the slot sides thus determine the range of spinal rod diameters that are accommodated by the spinal rod reception slots and therefore the spinal rod connector.

The spinal rod connector may be fashioned as a polyaxial spinal bone screw head for receiving a spinal rod therein. In another form, the spinal rod connector may be fashioned as an adjunct connector to a spinal screw assembly or as a spinal rod hook assembly.

The present spinal rod connectors accommodate a range of spine or spinal rods of various diameters. In one form, the spinal rod connector has a spinal rod reception range of 1.35 millimeters (mm). Thus, in an exemplary form and in keeping with the exemplary range of 1.35 mm, a spinal rod connector is configured to accommodate spine rods having a diameter ranging from 5.0 mm to 6.35 mm. The spinal rod connector can therefore accommodate three spinal rods, one with a diameter of 5.0 mm, one with a diameter of 5.5 mm, and one with a diameter of 6.35 mm with the 1.35 mm spinal rod reception range. Of course, the slots may be sized, configured and/or angled or tapered to accept any range of diameters of spinal rods.

The present spinal rod connector is formed of one or more biocompatible materials suitable for spine implants. Various sizes of the spinal rod connector may be manufactured for accommodating varying ranges of diameters of spinal rods.

### BRIEF DESCRIPTION OF THE DRAWINGS

The above mentioned and other features, advantages and objects of this invention, and the manner of attaining them, will become apparent and the invention itself will be better understood by reference to the following description of embodiments of the invention taken in conjunction with the accompanying drawings, wherein:

FIG. 1 is a top perspective view of a spinal rod connector embodied as a polyaxial spinal rod screw head fashioned in accordance with the present principles;

FIG. 2 is a side view of the polyaxial spinal rod screw head of FIG. 1;

FIG. 3 is a top plan view of the polyaxial spinal rod screw head of FIG. 1 taken along line 3-3 of FIG. 2;

FIG. 4 is a sectional view of the polyaxial spinal rod screw head of FIG. 1 taken along line 4-4 of FIG. 3;

FIG. 5 is a sectional view of the polyaxial spinal rod screw head of FIG. 1 taken along line 5-5 of FIG. 3;

FIG. 6 is a sectional view of the polyaxial spinal rod screw head of FIG. 1 taken along line 6-6 of FIG. 3;

FIG. 7 is a partial sectional view of a portion of the polyaxial spinal rod screw head of FIG. 1 taken along circle 7-7 of FIG. 5;

FIG. 8 is an enlarged partial sectional view of a portion of the polyaxial spinal rod screw head of FIG. 1 taken along circle 8-8 of FIG. 7;

FIG. 9 is an enlarged partial sectional view of a portion of the polyaxial spinal rod screw head of FIG. 1 taken along circle 9-9 of FIG. 5;

FIG. 10 is an enlarged partial sectional view of a portion of the polyaxial spinal rod screw head of FIG. 1 taken along circle 10-10 of FIG. 4 particularly showing optional internal threading of a bore of the base of the spine screw head;

FIG. 11 is a side view of the polyaxial spinal rod screw head of FIG. 1 showing the reception of a 5.0 millimeter spinal rod therein;

FIG. 12 is a side view of the polyaxial spinal rod screw head of FIG. 1 showing the reception of a 5.5 millimeter spinal rod therein;

FIG. 13 is a side view of the polyaxial spinal rod screw head of FIG. 1 showing a 6.0 millimeter spinal rod therein;

FIG. 14 is a side perspective view of a spinal rod connector embodied as a spinal rod connector hook for a spinal assembly and showing reception of a spinal rod therein; and

FIG. 15 is a front view of the spinal rod hook connector of FIG. 14 showing reception of a spinal rod therein.

Like reference numerals indicate the same or similar parts throughout the several figures.

A description of the features, functions and/or configuration of the components depicted in the various figures will now be presented. It should be appreciated that not all of the features of the components of the figures are necessarily described. Some of these non discussed features as well as discussed features are inherent from the figures. Other non discussed features may be inherent in component geometry and/or configuration.

### DETAILED DESCRIPTION OF EMBODIMENTS OF THE INVENTION

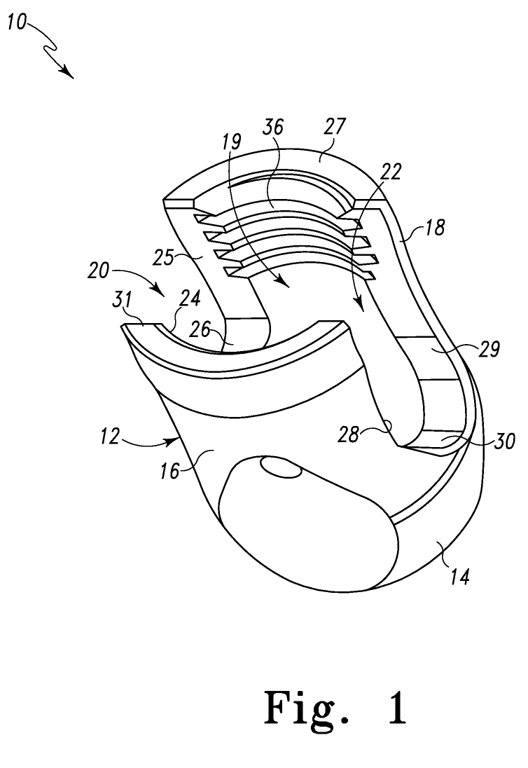

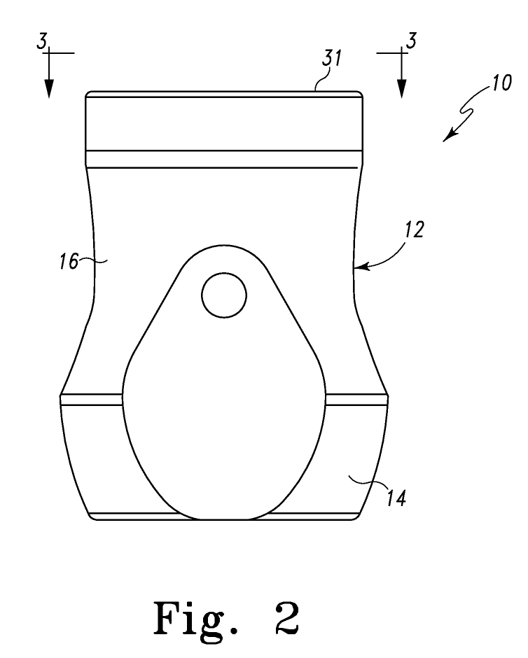

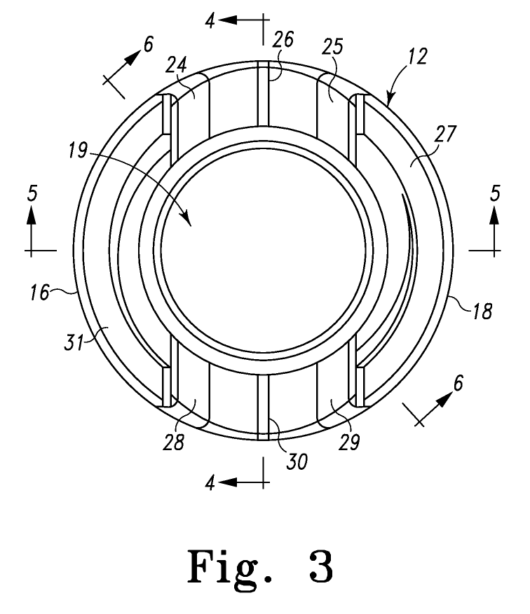

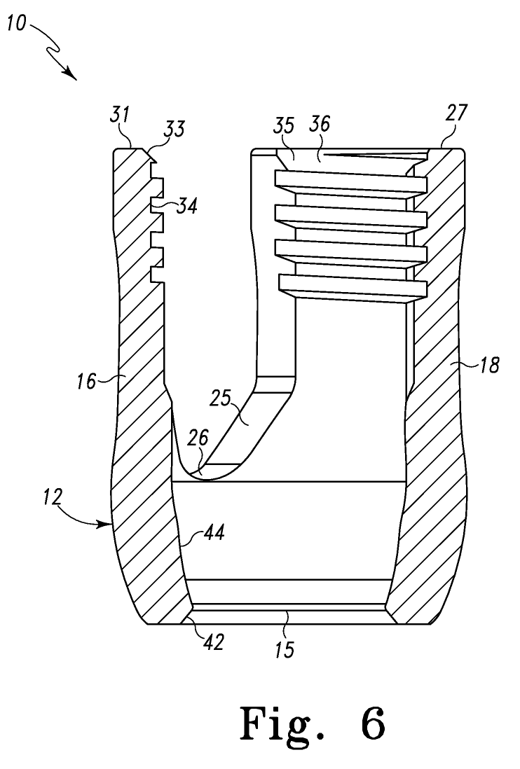

FIGS. 1-10 depict various views of a spinal rod connector embodied as a polyaxial spinal rod screw head or spinal rod holder for a spine screw or spine screw assembly generally designated 10 (and hereinafter, collectively, “spinal rod screw head 10”) fashioned in accordance with the present principles. The spinal rod screw head 10 is what is generally known as a tulip head, but other general configurations that utilize the present principles may, of course, be made. The spinal rod screw head 10 is used to hold a spinal rod relative to and on a bone screw (not shown) that is attached to a vertebra (not shown). As such, the spinal rod screw head 10 may be part of a spinal rod screw assembly.

The spinal rod screw head 10 is formed by a body 12 having a generally rounded and angled base 14. A first sidewall 16 extends axially from the base 14 at one side thereof while a second sidewall 18 extends axially from the base 14 at another side thereof. The first and second sidewalls 16, 18 are disposed on the base 14 generally opposite on another, each one of which is generally arc-shaped. The interior configuration of the first and second sidewalls 16, 18 define an interior area or space 19. The interior 19 is generally cylindrical in shape and thus may be considered as defining a bore in the body 12. The base 14 has an axial bore 15 therein that is in communication with the interior 19.

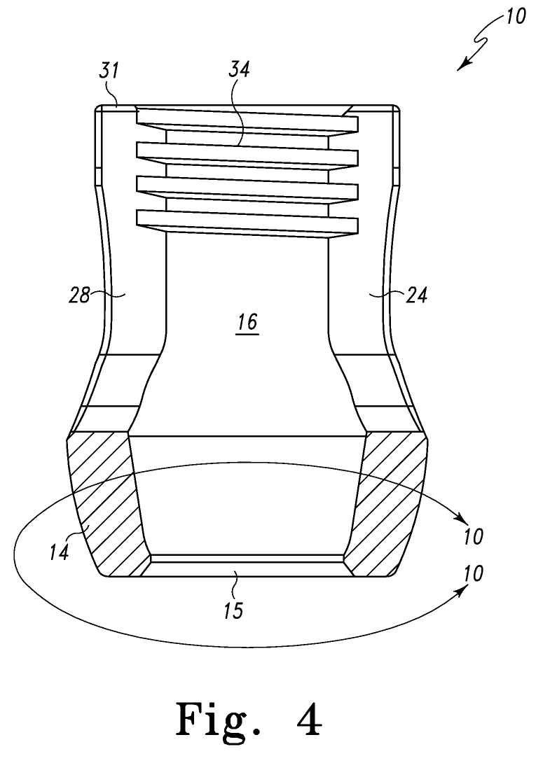

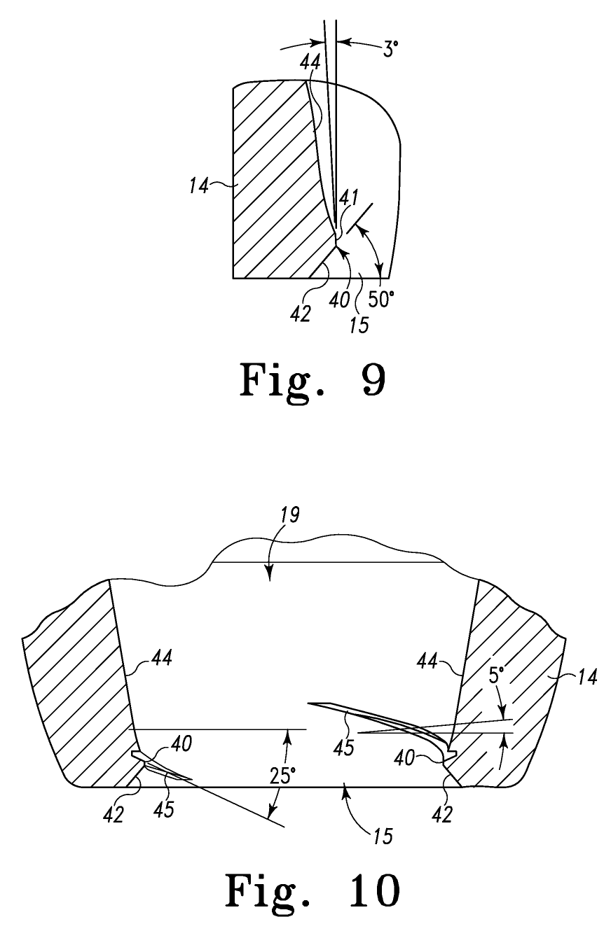

As best seen in FIGS. 4-6, the axial bore varies in diameter from the exterior of the base 14 to the interior 19 of the body 12; narrowing from the exterior to the interior thereof. The reduction in diameter of the bore 15 defines an annular rim or ridge 40 that extends about the lower portion of the interior 19. The bore 15 is thus slanted, angled or tapered inwardly from the exterior diameter of the bore 15 to the interior diameter of the bore 15 (i.e. rim 40). As best seen in FIG. 9, the slanting or angle of the bore 15 defines an annular slanted, angled or tapered surface 42 that extends between the exterior diameter of the bore 15 and the rim 40. In the form presented in the figures, the surface 42 has an angle of 50° relative to the bottom of the base 14. The rim 40 has an angled face 41 and as shown in the present embodiment has a 3° angled face 41. As shown in FIG. 10, the inner annular surface 44 of the lower (base) portion of the interior 19 arcs upwardly and outwardly from the rim 40. The described configuration of the bore 15 and the lower portion of the interior 19 allows pivotal retention of the head of a bone screw (not shown) while allowing the threaded shank of the bone screw (not shown) to extend therethrough. FIG. 10 also depicts optional threading 45 on the interior surfaces of the body 12 and particularly extending from the angled surface 42 of the bore 15 to the interior surface 44 of the interior 19.

The inner surface of the first sidewall 16 has threads or threading 34 that extends from an upper surface 31 to a distance axially downward. The threads 34 are slightly, downwardly angled. The threading 34 begins from an inwardly tapered annular surface 33 extending from the upper surface 31. As best seen in FIG. 8, the threads 34 start at a 3° depth relative to the upper surface 32 and end at a 15° depth such that the treading 34 are disposed at a 12° pitch. The inwardly tapering surface 33 and beginning of the threads 34 is at a 45° angle relative to the upper surface 31. The inner surface of the second sidewall 18 has threads or threading 36 that extends from an upper surface 37 to a distance axially downward. The threads 36 are slightly, downwardly angled in like manner to the threads 34 of the first sidewall 16. The threads 36 are slightly, downwardly angled in like manner to the threads 34 of the first sidewall 16. Likewise, the threads 36 begin from an inwardly tapered annular surface 35 extending from the upper surface 37. The threads 36 have the same characteristics as the threads 34 of the first sidewall 16 as explained above with reference to FIG. 8.

A first slot 20 is defined between one side of the first sidewall 16 and one side of the second sidewall 18. The first slot 20 extends a distance axially downward from one side of the top surfaces 31 and 27 to a bottom 26 of the slot 20 and is generally, but not necessarily, V-shaped. A second slot 22 is defined between another side of the first sidewall 16 and another side of the second sidewall 18. The second slot 22 extends a distance axially downward from another side of the top surfaces 31 and 27 to a bottom 30 of the slot 22 and is generally, but not necessarily, V-shaped. The first and second slots 20, 22 are configured to receive a range of varying diameter spine rods therein and together define a spine rod seat. The first and second slots 20, 22 are preferably, but not necessarily, disposed diametrically opposite one another on the body 12.

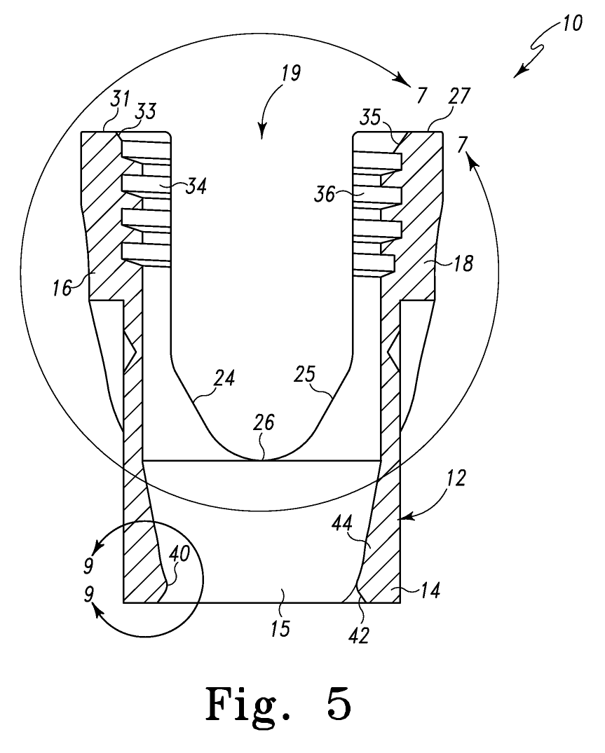

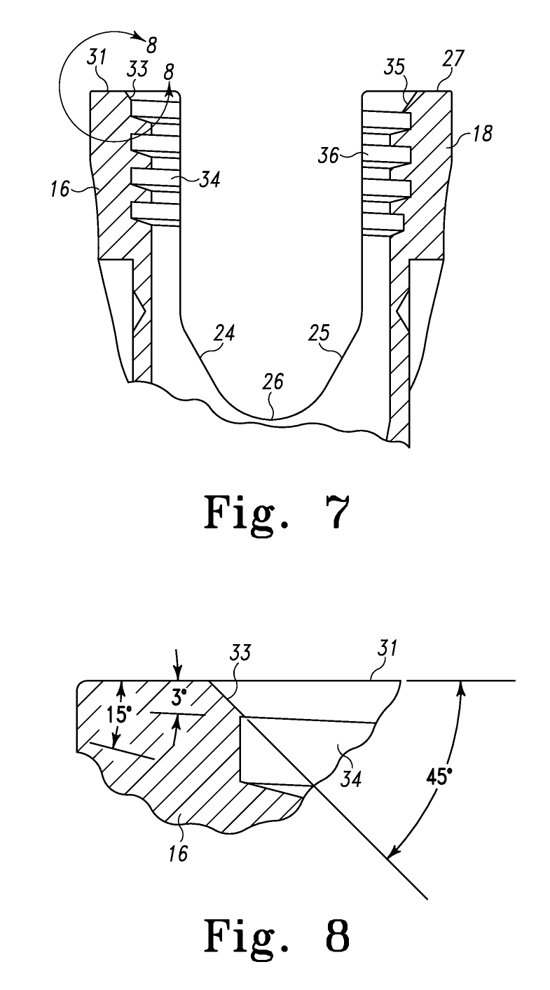

The configuration of the first slot 20 is defined by a first side surface 24 of one side of the first sidewall 16 and a second side surface 25 of one side of the second sidewall 18 that join at the bottom 26 thereof. As best seen in FIGS. 5 and 7, the first and second side surfaces 24, 25 of the slot 20 angle, slant or taper inwardly toward each other, meeting at the bottom 26. The angle or taper of the two side surfaces 24, 25 provide a varying wedge or wedging feature for receiving a range of varying spinal rods.

The second slot 22 is defined by a first side surface 28 of another side of the first sidewall 16 and a second side surface 29 of another side of the second sidewall 18 that join at the bottom 30 thereof. As best seen in FIGS. 5 and 7, the first and second side surfaces 28, 28 of the slot 22 angle, slant or taper inwardly toward each other, meeting at the bottom 30. The angle or taper of the two side surfaces 28, 29 provide a varying wedge or wedging feature for receiving a range of varying spinal rods.

The degree and or length of the angled side surfaces 24, 25 and 28, 29 of the slots 20 and 22 respectively, determines the range of varying diameter spinal rods that are useable with the particular screw head 10. In the embodiment shown, the slots 20 and 22 are configured to accept an approximate 1.5 mm range of diameters of spinal rods. The slot configuration and dimensions thus defines the size and dimensions of spinal rods that may be received and retained in and by the present screw head 10.

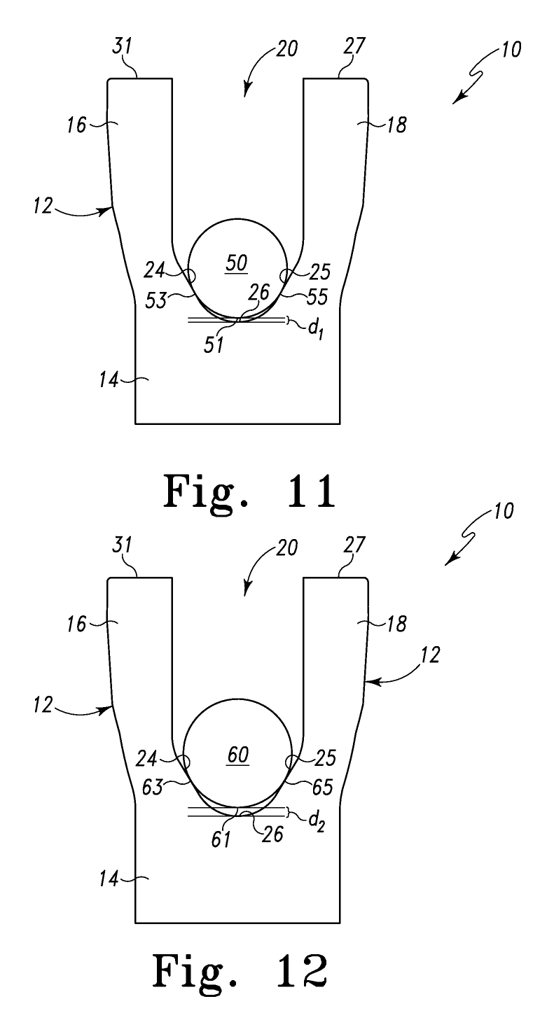

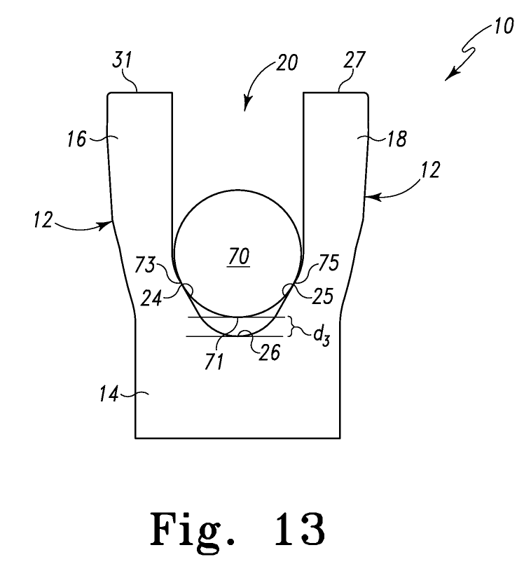

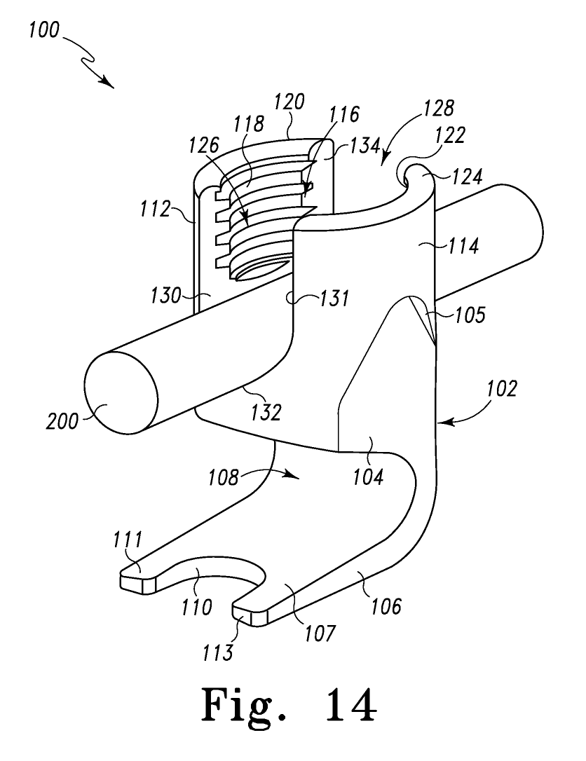

FIGS. 11-13 show side plan views of the present screw head accommodating spine rods of three (3) different diameters. Particularly, FIG. 11 shows a 5.0 mm diameter spinal rod 50 received in the slot 20 of the screw head 10. FIG. 12 shows a 5.5 mm diameter spinal rod 52 received in the slot 20 of the screw head 10. FIG. 13 shows a 6.35 mm diameter spinal rod 54 received in the slot 20 of the screw head 10. While not shown in the figures, it should be appreciated that the spine rod is received in the slot 22 of the head 10 in the same manner that the spine rod is received in the slot 20 thereof. Together, slots 20 and 22 form a spinal rod seat for receiving a portion of a spinal rod. The spinal rod seat receives two portions of the spinal rod. In use, the spinal rod is locked into the screw head slots and thus into the screw head at or approximate to the positions shown.

In FIG. 11 it can be seen that the 5.0 mm spine rod 50 is wedged low within the slot 20 such that a lower point 51 of the spine rod 50 is proximate the bottom 26 of the slot 20. The diameter of the spine rod 50 defines a lower end of a range of varying diameter spine rods that can be accommodated by the spine rod screw head 10. The spine rod 50 settles into a lower wedging position that defines a minimum distance d₁ between the lower point 51 of the spine rod 50 and the bottom 26 of the slot 20. A point or arc 53 on one side of the spine rod 50 abuts a lower portion of the narrowing side 24 of the slot 20. A point or arc 55 on another side of the spine rod 50 abuts a lower portion of the narrowing side 25 of the slot 20. In this manner, the spine rod 50 is wedged low into the slot 20 and thus the spine screw head 10 by the narrowing configuration of the slot 20.

In FIG. 12 it can be seen that the 5.5 mm spine rod 60 is wedged within the slot 20 at an intermediate wedging position that is higher up relative to the bottom 26 of the slot 20 than the lower wedging position of the smallest diameter spinal rod 50 as shown in FIG. 11. The diameter of the spine rod 60 is within the upper and lower ends of the range of varying diameter spine rods that can be accommodated by the spine rod screw head 10. An intermediate distance d₂ is thus defined between the lower point 61 of the spine rod 60 and the bottom 26 of the slot 20. A point or arc 63 on one side of the spine rod 60 abuts an intermediate portion of the narrowing side 24 of the slot 20. A point or arc 65 on another side of the spine rod 60 abuts an intermediate portion of the narrowing side 25 of the slot 20. In this manner, the spine rod 60 is wedged intermediate into the slot 20 and thus the spine screw head 10 by the narrowing configuration of the slot 20.

In FIG. 13 it can be seen that the 6.35 mm spine rod 70 is wedged within the slot 20 at an upper wedging position that is higher up relative to the bottom 26 of the slot 20 than the intermediate wedging position of the intermediate diameter spinal rod 70 as shown in FIG. 12. An upper distance d₃ is thus defined between the lower point 71 of the spine rod 70 and the bottom 26 of the slot 20. A point or arc 73 on one side of the spine rod 70 abuts an upper portion of the narrowing side 24 of the slot 20. A point or arc 75 on another side of the spine rod 70 abuts an upper portion of the narrowing side 25 of the slot 20. In this manner, the spine rod 70 is wedged higher into the slot 20 and thus the spine screw head 10 by the narrowing configuration of the slot 20. The diameter of the spine rod 70 defines an upper end of the range of varying diameter spine rods that can be accommodated by the spine rod screw head 10.

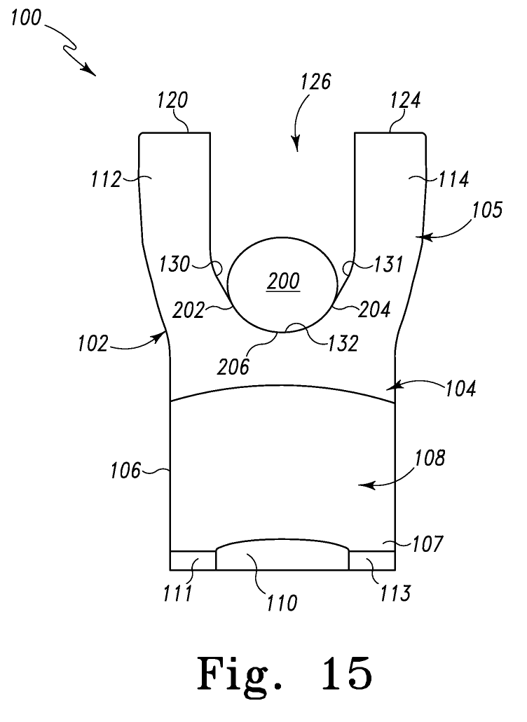

FIGS. 14 and 15 depict two views of another spinal rod connector embodied as a spinal rod connector hook for a spinal assembly generally designated 100 (and hereinafter, “spinal rod connector 100”) fashioned in accordance with the present principles. The spinal rod connector 100 is used to hold a spinal rod relative to and on a bone screw (not shown) that is attached to a vertebra (not shown) or to another type of a spinal rod assembly (not shown). As such, the spinal rod connector 100 may be part of a spinal rod screw assembly.

The spinal rod connector 100 is formed by a body 102 having a base 104 and a head 105. The head is defined by a first sidewall 112 that extends axially from the base 104 at one side thereof while a second sidewall 114 extends axially from the base 104 at another side thereof. The first and second sidewalls 112, 114 are disposed on the base 104 generally opposite on another, each one of which is generally arc-shaped. The interior configuration of the first and second sidewalls 112, 114 define an interior area or space 116. The interior 116 is generally cylindrical in shape and thus may be considered as defining a bore in the body 102.

The inner surface of the first sidewall 112 has threads or threading 118 that extends from an upper surface 120 to a distance axially downward. The threads 118 may be slightly, downwardly angled. The threading 118 begins from an inwardly tapered annular surface extending from the upper surface 120 in like manner as the head 10. The inner surface of the second sidewall 114 has threads or threading 122 that extends from an upper surface 124 to a distance axially downward. The threads 122 may be slightly, downwardly angled in like manner to the threads 118 of the first sidewall 112. The threads 118 and 122 have the same characteristics as the threads of the first and second sidewalls of the spinal rod screw head 10 as explained above with reference to FIG. 8.

A first slot 126 is defined between one side of the first sidewall 112 and one side of the second sidewall 114. The first slot 126 extends a distance axially downward from one side of the top surfaces 120, 124 to a bottom 132 of the slot 126 and is generally, but not necessarily, V-shaped. A second slot 128 is defined between another side of the first sidewall 112 and another side of the second sidewall 114. The second slot 128 extends a distance axially downward from another side of the top surfaces 120 and 124 to a bottom (not seen in the figures) of the slot 128 and is generally, but not necessarily, V-shaped. The first and second slots 126, 128 are configured to receive a range of varying diameter spine rods therein and together define a spine rod seat. The first and second slots 126, 128 are preferably, but not necessarily, disposed diametrically opposite one another on the body 102.

The configuration of the first slot 126 is defined by a first side surface 130 of one side of the first sidewall 112 and a second side surface 131 of one side of the second sidewall 114 that join at the bottom 132 thereof. As best seen in FIG. 15, the first and second side surfaces 130, 131 of the slot 126 angle, slant or taper inwardly toward each other, meeting at the bottom 132. The angle or taper of the two side surfaces 130, 131 provide a varying wedge or wedging feature for receiving a range of varying spinal rods.

The second slot 128 is defined by a first side surface 134 of another side of the first sidewall 112 and a second side surface (not seen) of another side of the second sidewall 114 that join at the bottom (not seen) thereof both in like manner to the second side surface 131 and bottom 132 of the first slot 126. The first and second side surfaces of the slot 128 angle, slant or taper inwardly toward each other, meeting at the bottom thereof. The angle or taper of the two side surfaces of the slot 128 provide a varying wedge or wedging feature for receiving a range of varying spinal rods in like manner to the slot 126 and as described above.

The base 104 includes a hook or hook portion 106 that angles from a bottom portion of the base 104. The hook 106 particularly has a generally 90° angle as taken from a longitudinal axis of head 105 (and bore 116). The hook 106 thus defines an arched or curved interior 108 configured or fashioned to be received about and/or hook or attach onto a spinal rod or other spinal component or assembly. The hook 106 further has an elongated portion 107 that is configured to extend underneath the spinal rod or other spinal component or assembly. A slot or cutout 110 is formed in the end of the elongated portion 107 thereby defining a first flange 111 and a second flange 113. The cutout 110 is generally arched and is thus configured to be received under a bone screw head or other spinal rod component or assembly.

It should be appreciated that spinal rod connectors may be fashioned in various sizes to accommodate varying ranges of spinal rods. In all cases though, the spinal rod connectors are made from a bio-compatible material such as stainless steel or titanium. Other bio-compatible materials, or course, may be used.

While the invention has been illustrated and described in detail in the drawings and foregoing description, the same is to be considered as illustrative and not restrictive in character, it being understood that only preferred embodiments have been shown and described and that all changes and modifications that come within the spirit of the invention are desired to be protected.

What is claimed is:

1. A spinal rod connector for a spinal rod bone screw assembly, the spinal rod connector comprising:
a body defining an upper end, a lower end and an annular side wall between the upper end and the lower end;
a first slot formed in the annular side wall, the first slot defined by a first side wall, a second sidewall and a bottom, the first and second side walls of the first slot angled inwardly towards each other proximate the bottom of the first slot to meet at the bottom of the first slot; and
a second slot formed in the annular side wall, the second slot defined by a first side wall, a second side wall and a bottom, the first and second side walls of the second slot angled inwardly towards each other proximate the bottom of the second slot to meet at the bottom of the second slot.

2. The spinal rod connector of claim 1, wherein the first and second slots are formed in the annular side wall diametrically opposite one another.

3. The spinal rod connector of claim 1, wherein the first and second angled side walls of the first and second slots are each sized to accept a range of diameters of spinal rods.

4. The spinal rod connector of claim 3, wherein the range of diameters of spinal rods comprises 1.35 mm.

5. The spine rod connector of claim 1, further comprising a hook formed axially below the lower end, the hook sized to be received on an installed spinal rod.

6. A spinal rod connector for a spinal rod bone screw assembly, the spinal rod connector comprising:
a body defining an upper end, a lower end and an annular side wall between the upper end and the lower end;
a bore extending from the upper end to the lower end, the bore sized at the upper end to allow a head of a bone screw to pass therethrough and sized at the lower end to retain the head of the bone screw;
a first slot formed in the annular side wall, the first slot defined by a first side wall, a second sidewall and a bottom, the first and second side walls of the first slot angled inwardly towards each other proximate the bottom of the first slot to meet at the bottom of the first slot; and
a second slot formed in the annular side wall, the second slot defined by a first side wall, a second side wall and a bottom, the first and second side walls of the second slot angled inwardly towards each other proximate the bottom of the second slot to meet at the bottom of the second slot.

7. The spinal rod connector of claim 6, wherein the lower end of the bore is tapered.

8. The spinal rod connector of claim 7, wherein the tapered lower end of the bore provides polyaxial movement of the rod connector relative to the bone screw head.

9. The spinal rod connector of claim 6, wherein the first and second slots are formed in the annular side wall diametrically opposite one another.

10. The spinal rod connector of claim 6, wherein the first and second angled side walls of the first and second slots are each sized to accept a range of diameters of spinal rods.

11. The spinal rod connector of claim 10, wherein the range of diameters of spinal rods comprises 1.35 mm.

12. The spinal rod connector of claim 11, wherein the first and second slots are configured to accept spinal rods of a diameter of 5.0 mm up to spinal rods of a diameter of 6.35 mm.

13. The spinal rod connector of claim 1, wherein the body includes threads on inner side walls of the annular side wall of the bore.

14. A spinal rod connector for receiving a spinal rod and retaining the spinal rod onto a bone screw, the spinal rod connector comprising:
a generally cylindrical body defining an upper end, a lower end and an annular side wall between the upper end and the lower end;
a generally cylindrical bore extending from the upper end of the body to the lower end of the body, the bore sized at the upper end to allow a bone screw to pass therethrough but sized at the lower end to retain a head of the bone screw while allowing a shank of the bone screw to pass therethrough;
a first slot formed in the annular side wall, the first slot defined by a first side wall, a second sidewall and a bottom, the first and second side walls of the first slot angled inwardly towards each other proximate the bottom of the first slot to meet at the bottom of the first slot; and
a second slot formed in the annular side wall, the second slot defined by a first side wall, a second side wall and a bottom, the first and second side walls of the second slot angled inwardly towards each other proximate the bottom of the second slot to meet at the bottom of the second slot.

15. The spinal rod connector of claim 14, wherein the first and second slots are formed in the annular side wall diametrically opposite one another.

16. The spinal rod connector of claim 14, wherein the first and second angled side walls of the first and second slots are each sized to accept a range of diameters of spinal rods.

17. The spinal rod connector of claim 16, wherein the range of diameters of spinal rods comprises 1.35 mm.
